(19) United States
(12) Patent Application Publication
(10) Pub. No.: US 2025/0332392 A1
(43) Pub. Date: Oct. 30, 2025

(54) APPARATUSES AND METHODS FOR TREATMENT OF FEMALE URINARY INCONTINENCE

(71) Applicants: THE BOARD OF REGENTS OF THE UNIVERSITY OF TEXAS SYSTEM, Austin, TX (US); OAKCREST INSTITUTE OF SCIENCE, Monrovia, CA (US)

(72) Inventors: Kathleen Vincent, League City, TX (US); Melinda Sheffield-Moore, New Braunfels, TX (US); Marc Baum, Altadena, CA (US); John Moss, Sierra Madre, CA (US)

(21) Appl. No.: 19/130,969

(22) PCT Filed: Nov. 20, 2023
(86) PCT No.: PCT/US2023/080577

§ 371 Date: May 19, 2025

Related U.S. Application Data
(60) Provisional application No. 63/426,605, filed on Nov. 18, 2022.

(51) Int. Cl. A61M 31/00; A61F 6/08

(52) U.S. Cl. CPC A61M 31/002; A61F 6/08; A61M 2210/1475

(57) ABSTRACT

In one embodiment, an intravaginal apparatus includes a drug delivery device configured for insertion into and residence within the vagina of a subject adjacent to the subject's pelvic floor, the drug delivery device further being configured to deliver one or more drugs to tissues within the vagina, the one or more drugs including a PDE5 inhibitor.

### CROSS-REFERENCE TO RELATED APPLICATION

This application claims priority to co-pending U.S. Provisional Application Ser. No. 63/426,605, filed Nov. 18, 2022, which is hereby incorporated by reference herein in its entirety.

### BACKGROUND

Stress urinary incontinence (UI) is a common problem in women and is primarily associated with weakness or loss of support of the pelvic floor muscles. Approximately 25% of young women, 50% of middle-aged and postmenopausal women, and 75% of older women experience some involuntary urine loss with up to 93% attributed to stress UI or “mixed” UI, which is a combination of stress UI and urge UI, which is also referred to as overactive bladder.

Current noninvasive treatments for UI include pelvic floor physical therapy and topical estrogen. Unfortunately, these treatments have provided little to no long-term symptom relief. Most symptomatic women present with advanced cases where a pessary and/or surgery are the only options. In some cases, UI is not responsive to pessary use and, therefore, surgical management is the only option. Currently, there is a 13.6% lifetime risk of surgery, and recurrence after surgery is common and comes with surgical risks that increase with increasing age.

In view of the above discussion, it can be appreciated that it would be desirable to have alternative, more effective, treatments for UI.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1A is a perspective view of an embodiment of an intravaginal ring for treating female urinary incontinence.

FIG. 1B is a partial cross-sectional view of the intravaginal ring of FIG. 1A.

FIG. 2A is a perspective view of an embodiment of a pessary for treating female urinary incontinence.

FIG. 2B is a cross-sectional perspective view of the pessary of FIG. 2A.

FIG. 3A is a top perspective view of another embodiment of a pessary for treating female urinary incontinence.

FIG. 3B is a bottom perspective view of the pessary of FIG. 3A.

FIG. 4 is a perspective view of another embodiment of a pessary for treating female urinary incontinence.

FIG. 5 is a perspective view of another embodiment of a pessary for treating female urinary incontinence.

FIG. 6A top perspective view of another embodiment of a pessary for treating female urinary incontinence.

FIG. 6B is a cross-sectional side perspective view of the pessary of FIG. 6A.

FIG. 7 is a perspective view of another embodiment of a pessary for treating female urinary incontinence.

FIG. 8 is a perspective view of another embodiment of a pessary for treating female urinary incontinence.

FIG. 9 is a perspective view of another embodiment of a pessary for treating female urinary incontinence.

FIG. 10 is a perspective view of another embodiment of a pessary for treating female urinary incontinence.

### DETAILED DESCRIPTION

As described above, current treatments for female urinary incontinence (UI) are often ineffective and/or invasive. Disclosed herein are apparatuses and methods for treating UI that are less invasive and more effective. In some embodiments, an intravaginal apparatus, such as an intravaginal ring and/or pessary, is used to deliver one or more therapeutic agents, such as PDE5 inhibitors (e.g., sildenafil), to increase blood flow and induce muscle anabolism to the muscles of the pelvic floor to provide relief from the symptoms of stress, urge, and/or mixed UI in women. The therapeutic agents are delivered directly to the muscles of the pelvic floor and, in embodiments in which the apparatus includes a pessary, structural support is also provided to those muscles.

In the following disclosure, various specific embodiments are described. It is to be understood that those embodiments are example implementations of the disclosed inventions and that alternative embodiments are possible. Such alternative embodiments include hybrid embodiments that include features from different disclosed embodiments. All such embodiments are intended to fall within the scope of this disclosure.

In a clinical trial performed by the inventors, women taking oral sildenafil exhibited a decrease in the number of UI episodes per day, but experienced side effects, including flushing, visual changes, and gastrointestinal changes. In view of this, the inventors determined to develop a way to administer sildenafil directly to the muscles and structures of the pelvic floor using an intravaginal apparatus. Disclosed in the discussion that follows are example embodiments of intravaginal apparatuses that can be used for treating urinary incontinence.

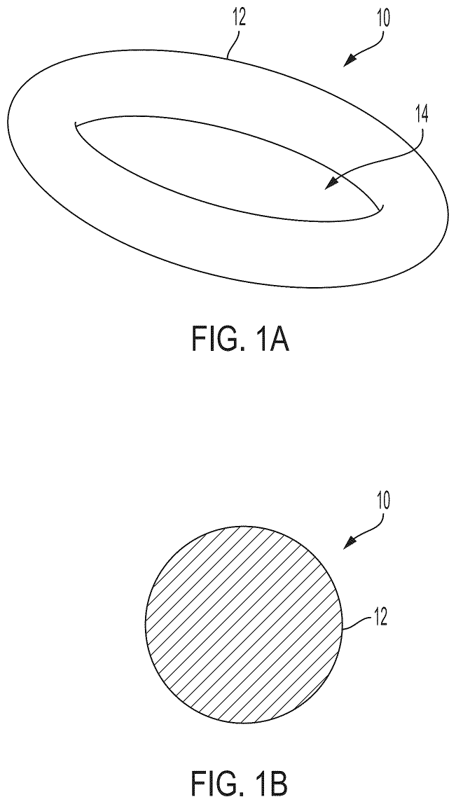

FIGS. 1A and 1B illustrate a first example intravaginal apparatus for the administration of one or more therapeutic agents (“drugs”), including PDE5 inhibitors such as sildenafil, to a patient. As is apparent from the figure, the apparatus is configured as an intravaginal ring (IVR) 10 intended for insertion into and fixation within the vagina immediately adjacent to the pelvic floor muscles. As shown in the figure, the ring 10 comprises a continuous toroidal body 12 that defines an inner opening 14. The ring body 12 can be made of a soft, flexible polymeric material, such as silicone, ethylene-vinyl acetate (EVA) copolymer, polyurethane, polyurethane-siloxane copolymer, polyester, polyamide, polyolefin, styrenic block copolymer, and latex, and can have an inner diameter of approximately 1 to 7 cm, an outer diameter of approximately 3 to 8 cm, and a cross-sectional diameter of approximately 2 to 12 mm.

The intravaginal ring 10 is configured to deliver one or more drugs to the pelvic floor and, therefore, may more generally be referred to as a drug delivery device. As noted above, the drugs delivered by the ring 10 can include one or more PDE5 inhibitors. Example PDE5 inhibitors include sildenafil (e.g., Viagra™, Pfizer), vardenafil (e.g., Levitra™ and Staxyn™, Bayer/GlaxoSmithKline), tadalafil (e.g., Cialis™, Eli Lilly), and avanafil (e.g., Stendra™, Vivus). The drugs can be comprised by the ring 10 in any manner with which the drugs can be effectively delivered to the surrounding tissues within the vagina. In one embodiment, the ring body 12 can be infused with the drugs. For example, the drugs can be mixed with the material from which the body 12 is made. Optionally, an unmedicated coating can be applied to the outer surface of the body 12 to modify rate of drug delivery. In another embodiment, the drugs can be contained within a coating that is applied to the outer surface of the body 12. In a further embodiment, the body 12 can comprise cavities in which the drugs can be provided. For example, one or more drugs can be provided within the cavities in the form of a solid filler material, a powder, a liquid, or a tablet or capsule.

Regardless of the manner in which the one or more drugs are provided on or within the ring 10, they can be delivered directly to the muscles and structures of the pelvic floor for enhanced therapeutic effect. In some embodiments, the ring 10 can be discarded once it has been completely or nearly depleted and can no longer deliver the desired dosage to the vaginal tissues.

Notably, other drugs can be delivered by the intravaginal ring 10, either in addition to or in lieu of one or more PDE5 inhibitors. Examples include estrogenic compounds (e.g., estradiol), progestin compounds (e.g., progesterone), androgenic compounds (e.g., testosterone), and anti-incontinence medications, such as tolterodine and oxybutynin.

Once inserted and properly positioned within the vagina, the intravaginal ring 10 can be left in place for an extended period of time, such as multiple days, weeks, or months. By way of example, the ring 10 can be configured for 30 to 90 days of residence and continuous drug release and delivery within the vagina. The ring 10 can be configured to deliver the one or more drugs at particular delivery rates, which are often expressed as a quantity (e.g., a mass) per day (i.e., 24-hour period). By way of example, the ring 10 can be configured to deliver a PDE5 inhibitor such as sildenafil at a generally constant rate of approximately 1 μg/day to 20 mg/day for a given period of time, such as 30 days. In some embodiments, the ring 10 has a PDE5 inhibitor delivery rate of approximately 10 μg/day to 0.5 mg/day. In other embodiments, the ring 10 has a PDE5 inhibitor delivery rate of approximately 0.5 to 3 mg/day. In further embodiments, the ring 10 has a PDE5 inhibitor delivery rate of approximately 3 to 20 mg/day. In still other embodiments, the ring 10 has a PDE5 inhibitor delivery rate of approximately 0.5 to 1 mg/day.

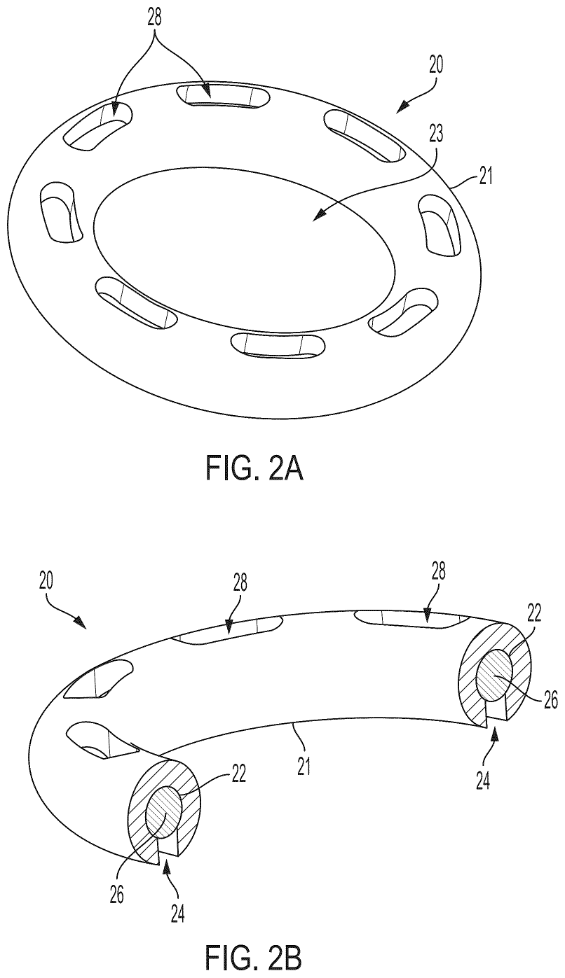

In addition to delivering drugs, the intravaginal apparatus can, in some embodiments, provide structural support to the pelvic organs. This can be achieved by combining a drug delivery device similar to the intravaginal ring 10 shown in FIG. 1 with a further intravaginal device that is specifically designed to provide structural support to the pelvic floor, bladder, and/or urethra. For instance, the further device can comprise a pessary that is configured to receive a separate drug delivery device, such as a ring. FIG. 2 illustrates a first example pessary 20 that can be used in such a manner.

Shown in FIG. 2A is a pessary 20 that, like the intravaginal ring 10, is also configured as a continuous ring. More particularly, the pessary 20 comprises a continuous toroidal body 21 that defines an inner opening 23. The body 21 can be made of a flexible polymeric material such as silicone, ethylene-vinyl acetate (EVA) copolymer, polyurethane, polyurethane-siloxane copolymer, polyester, polyamide, polyolefin, styrenic block copolymer, and latex. Notably, however, the material from which the pessary 20 is made can stiffer than the material from which the ring 10 is made so that the pessary is capable of providing structural support to the bladder and vaginal tissues. The pessary 20 can have an inner diameter of approximately 1 to 7 cm, an outer diameter of approximately 3 to 8 cm, and a cross-sectional dimension (e.g., diameter) of approximately 2 to 12 mm. Although the ring 10 and the pessary 20 are illustrated as being toroidal, the cross-sectional shapes of those apparatuses do not need to be circular. For example, the cross-sectional shapes of the apparatuses can be elliptical, rectangular, rectangular with rounded edges, hexagonal, etc. The cross-sectional shape can also be a complex shape that does not have a common geometrical name.

The pessary 20 differs from conventional pessaries at least in that, as shown most clearly in the cross-sectional view of FIG. 2B, it includes a toroidal inner cavity 22 that is accessible via a circular channel 24 formed within a bottom side of the pessary (in the orientation of FIG. 2B). The cavity 22 is configured to receive a separate drug delivery device 26, which, in some embodiments, can be a ring having a configuration similar to that of the intravaginal ring 10 of FIG. 1. To position the device 26 within the cavity 22 as depicted in FIG. 2B, the device can be passed through the channel 24 and inserted into the cavity 22 where it will remain during use of the pessary 20. In some embodiments, the cavity 22 has one or more dimensions (e.g., diameter) that are the same as or slightly smaller than those of the device 26 such that, when the device is inserted into the cavity, the device is securely held within the pessary 20 with a friction fit. While such a friction fit can be enough to ensure that the device 26 stays within the cavity 22 during pessary use, in some embodiments, additional securing means or elements, can be provided on or in the pessary 20 to ensure the device is retained within the cavity. For example, one or more prongs or tabs (not shown) can be provided that either grip the device 26 and/or block the channel 24.

During use, the one or more drugs comprised by the drug delivery device 26 can pass through the circular channel 24 and be received by the tissue adjacent the bottom side of the pessary 20 (in the orientation of FIG. 2). In addition, the one or more drugs can pass through openings 28 formed in the top side of the pessary 20 that, like the channel 24, extend to the inner cavity 22 to enable drugs comprised by the device 26 to be received by the tissues adjacent the top side of the pessary 20. Between the channel 24 and the openings 28, the one or more drugs of the device 26 can be delivered to all tissues that surround the pessary 20, including the muscles of the pelvic floor. By way of example, each opening 28 can have dimensions (e.g., length, width, or diameter) in the range of approximately 2 to 5 mm.

As noted above, the intravaginal ring 10 can be a disposable device that is discarded once it is no longer capable of delivering the desired dosage. The same can be true for the drug delivery device 26 that is used with the pessary 20. The pessary 20, however, can be designed for reuse. For example, a fresh drug delivery device 26 can be inserted into the pessary 20 and the pessary can then be inserted into the vagina. After a period of time, such as 30 days, the pessary 20 can be removed, the device 26 can be discarded, the pessary can be cleaned and disinfected, a new device 26 can be inserted into the pessary, and the pessary can be can be re-inserted into the vagina to continue treatment. In such a case, a patient can be provided with a pessary 20 and one or more devices 26. If treatment is to continue after all of the devices 26 have been depleted, the patient could then obtain further devices 26 from her physician or a pharmacy.

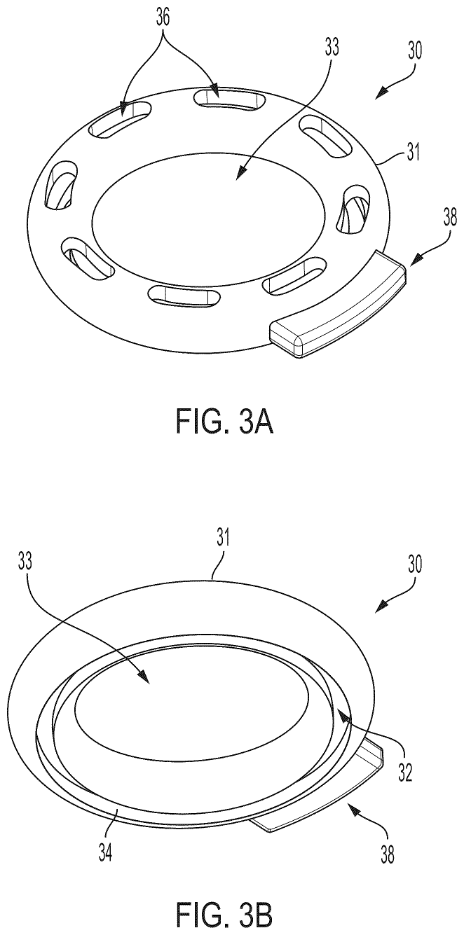

FIG. 3 illustrates another pessary 30 that can be used in conjunction with a drug delivery device, such as the ring 26 shown in FIG. 2B. The pessary 30 is similar in several ways to the pessary 20 shown in FIG. 2. Accordingly, the pessary 30 comprises a continuous toroidal body 31 that defines an inner opening 33. The body 31 can be made of a flexible polymeric material, such as those identified above for the pessary 20, and can have dimensions similar to those of the pessary 20. The pessary 30 also includes an inner cavity 32 that is configured to receive the drug delivery device, which can be passed through a circular channel 34. The pessary 30 is also provided with multiple openings 36 that enable the drugs comprised by the drug delivery device to be delivered to the vaginal tissues. Unlike the pessary 20, the pessary 30 further includes a urethral support element 38 that is configured to provide structural support to the patient's urethra to reduce incidence of incontinence. In the embodiment of FIG. 3, the support element 38 is configured as a rectangular tab that extends radially outward from a lateral side of the pessary 30.

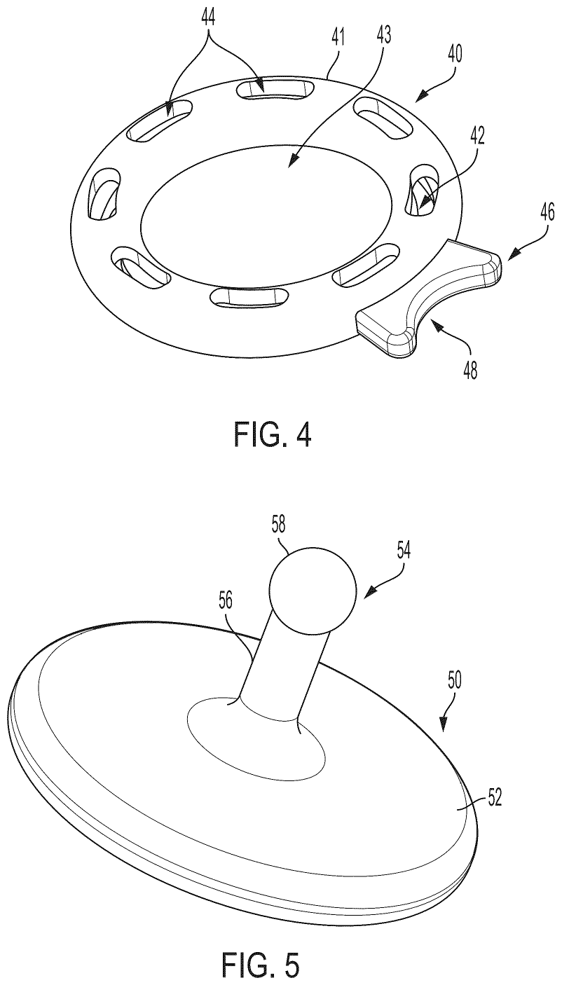

Although FIG. 3 illustrates a particular configuration for a urethral support element, it is noted that other configurations are possible. FIG. 4 shows an example alternative configuration. More particularly, FIG. 4 illustrates a further pessary 40 that, like the pessary 30, comprises a continuous toroidal body 41 that defines an inner opening 43. The pessary 40 includes an inner cavity 32 that is configured to receive a drug delivery device, which can be passed through a circular channel provided on a bottom side of the pessary (not visible in FIG. 4). In addition, the pessary 40 includes openings 44 that enable the drugs comprised by the drug delivery device to be delivered to the surrounding vaginal tissues. Furthermore, the pessary 40 includes a urethral support element 46 that extends radially outward from a lateral side of the pessary 40. The element 46 is also configured as a rectangular tab, but it includes an arcuate notch 48 formed in its distal edge. This notched element 46 is intended to support the urethra to prevent or decrease incontinence as would the element 38 shown in FIGS. 3A and 3B, but avoids applying undue pressure to the urethra, which could interfere with the patient's ability to urinate when desired.

FIG. 5 illustrates a pessary 50 that is configured as a Gellhorn pessary. As such, the pessary 50 comprises a disc-shaped body 52 having a circular or ellipsoid shape and a handle 54 that extends perpendicularly outward from the center of the body that can be used to grip the pessary during its insertion and removal. As with the pessaries discussed above, the pessary 50 can be made of a flexible polymeric material. The body 52 can have an outer dimension (e.g., diameter) in the range of approximately 3 to 9 cm and a maximum thickness (e.g., at the center of the body) of approximately 0.2 to 1.5 cm. As shown in the figure, the handle 52 can comprise a stem 56 that extends from the body 52 and a bulb or knob 58 that is positioned at the distal end of the stem. In one embodiment, the handle 54 is permanently attached to the body 52. In an alternative embodiment, the handle 54 can be removed from the body 52. In such an embodiment, the handle 54 can have external threads that mate with internal threads formed within the body 52 to enable attachment and removal. Alternatively, the handle 54 can be attached to the body 52 using separate fastening elements, such as one or more screws. As a further alternative, the handle 54 can be secured to the body 52 using a locking mechanism to enable a press-fit. In yet another embodiment, the knob 58 can be removed from the stem 56.

In some embodiments, the pessary 50 can have one or more drugs embedded in the material from which it is made so that the pessary itself acts as a drug delivery device. The drugs can be embedded throughout the entire volume of the pessary 50 or the drugs can be embedded in one or more discrete parts of the pessary, such as the outer edge of the body 52, the handle 54, the handle stem 56, the handle knob 58, or any combination thereof. Alternatively, the entirety or one or more portions of the pessary 50 can be coated with a material that includes the drugs. In embodiments in which a part of the pessary 50, such as the handle 54, can be disconnected from the body 52, that part can be replaced once the drugs it comprises have been depleted.

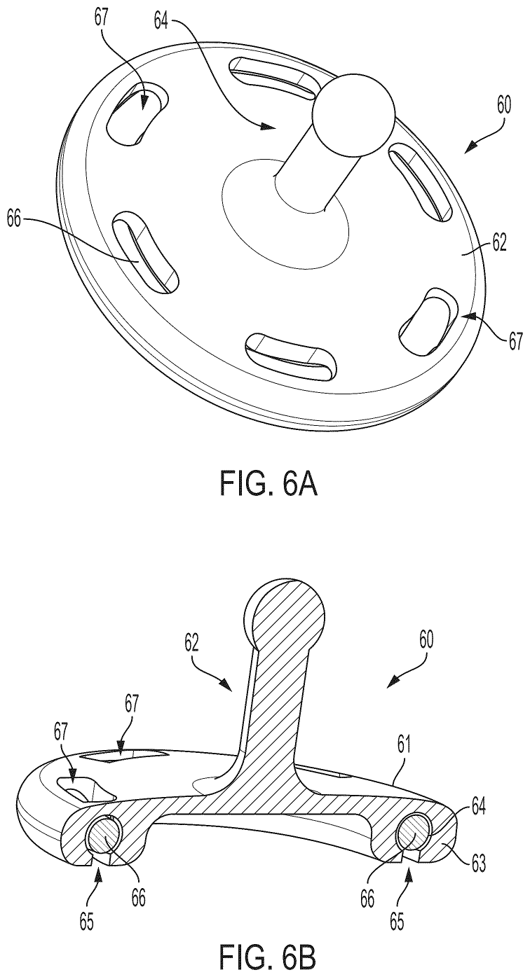

FIGS. 6A and 6B illustrate a pessary 60 that is a hybrid of the pessary 20 of FIG. 2 and the pessary 50 of FIG. 5. Like the pessary 50, the pessary 60 is configured as a Gellhorn pessary and, therefore, comprises a disc-shaped body 61 from which extends a handle 62. Unlike the pessary 50, however, the pessary's body 61 includes a continuous toroidal body or portion 63 that defines the outer edge of the body 61. As with the pessary 20, the toroidal portion 63 includes a toroidal inner cavity 64 accessible via a round channel 65 that is configured to receive a drug delivery device 66, such as a ring. In addition, the pessary 60 includes openings formed on the top side of the body 61 through which drugs comprised by the device 66 can pass.

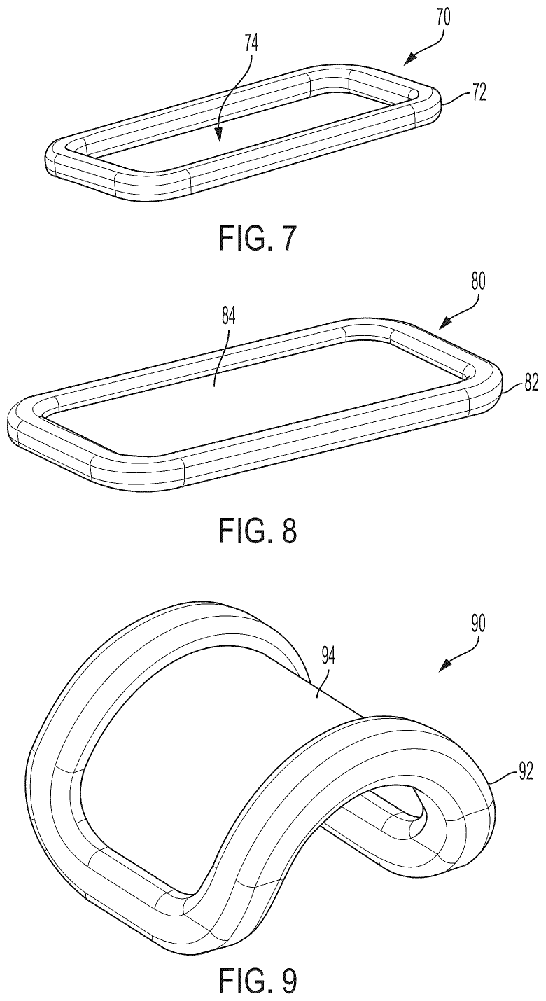

FIG. 7 illustrates a pessary 70 having a generally rectangular shape that is defined by a generally rectangular body 72 having rounded corners. Like the pessaries 20, 30, and 40 described above, the body 72 forms a continuous ring that defines an inner opening 74. As the pessary 70 is not used with a separate drug delivery device, the body 72 of the pessary 70 comprises one or more drugs that can be delivered to the vaginal tissues, those drugs contained within the material of the body, contained within a coating applied to the body, or both. In some embodiments, the body 72 can have a generally rectangular (e.g., square cross-section) or a generally circular or elliptical cross-section.

FIG. 8 illustrates another pessary 80 having a generally rectangular shape defined by a generally rectangular body 82 having rounded corners and defining an inner opening. In this embodiment, however, a central planar membrane 84 extends across the inner opening the between the inner edges of the body 82. In this embodiment, the body 82 comprises one or more drugs to be delivered, the membrane 84 comprises one or more drugs to be delivered, or both. When the membrane 84 is present, it can provide greater structural support than that provided by the open pessary 70 and, when provided with one or more drugs to be delivered, can deliver the drugs over a larger area.

FIG. 9 illustrates a pessary 90 having a similar configuration to that of the pessary 80 of FIG. 8 that includes both a generally rectangular body 92 and a central planar membrane 94 that extends between the inner edges of the body. In this embodiment, however, the body 92 and membrane 94 are curved along their lengths to form a generally arcuate structure for supporting the pelvic floor.

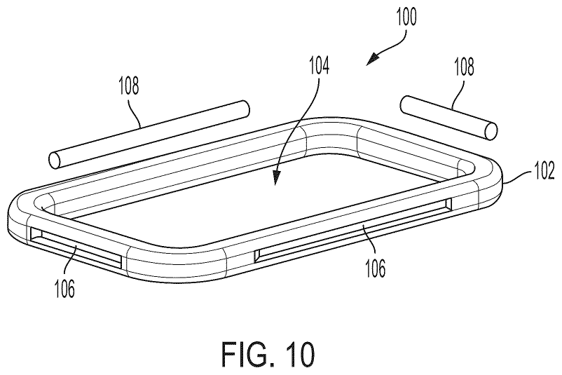

FIG. 10 illustrates yet another pessary 100 that is similar to the pessary 70 of FIG. 7. The pessary 100, therefore, includes a generally rectangular body 102 that forms a continuous ring and defines an inner opening 104. In the embodiment of FIG. 10, however, the body 102 includes discrete channels 106 that are configured to receive and hold rod-shaped drug delivery devices 108. The devices 108 can comprise one or more drugs to be delivered to the vaginal tissues and can be replaced once they are depleted in similar manner to the ring-shaped devices used in the embodiments shown in FIGS. 2-4.

In some embodiments, the intravaginal apparatus, such as a pessary, can be moldable into desired shapes so that the apparatus can be adjusted to fit the particular anatomy of the user. For example, the use of a moldable polymer can be employed or an embedded wire or coil (e.g. nitinol or other medical grade alloy, thermally set wire) can be incorporated into the apparatus to achieve this function. One example moldable polymer can be ethylene-co-vinyl acetate (EVA) having a vinyl acetate (VA) composition of 5-50%, preferably 8-40%, more preferably 15-28%, and ideally 28%. In other embodiments, the moldable polymer can be a polyimide; a poly (ethylene terephthalate glycol) (PETG); a thermoplastic polyurethane (TPU); a polyolefin including polyethylene (PE), polypropylene (PP), or polybutylene (PB); a vulcanized silicone rubber (RTV); or one or more thermosetting polymers based on ternary thiolene systems modified with urethane (UMTEN) or acrylate (AMTEN). In other embodiments, the moldable polymer can be a blend or laminate of two or more of the aforementioned polymers. In some embodiments, the shape of the intravaginal apparatus can be set using vacuum forming or pressure forming methods external to the vagina. Alternatively, an intravaginal apparatus can be molded by heating it to a temperature above the elastomer's glass transition temperature to make it malleable and then inserted into place within the vagina so that the shape is set when the pessary temperature cools in place.

A series of pharmacokinetic and safety studies were conducted in both women and sheep. The sheep studies were performed to exploit similarities in the vaginal physiology between sheep and humans, and because a clinical intravaginal ring was not available.

In the clinical trial, women were orally administered 20 mg of sildenafil 3 times a day for a month. The results from the trial showed that the drug concentrations within cervicovaginal (CVF) secretions were not dependent on the plasma drug concentrations and were higher than the plasma drug concentrations at the later timepoints after the last oral dose.

The sheep study was performed using four female Merino crossbred sheep. The sheep were given 10 to 15 mg of sildenafil (a dose equivalent to the dose per average weight of the subjects in the clinical trial) either orally 3 times a day or in a single intravenous (IV) dose. Plasma and CVF secretions were obtained from the sheep to determine the drug concentrations in each. The sheep were orally administered sildenafil and peak sildenafil plasma concentrations of 2 to 10 ng/ml were observed after 5 days, while sildenafil levels within the sheep's CVF were below the level of quantification. Within 15 minutes after a single IV dose was administered, sildenafil plasma levels peaked between 10⁴ and 10⁵ ng/ml and then rapidly dropped and stabilized after about an hour to approximately 10 ng/ml for the 24 hours during which plasma was collected. The CVF sildenafil levels were between 100 to 1,100 ng/gm at 1 and 4 hours after a single IV dose, but were below the level of quantification at 12 and 24 hours.

In the next phase of the study, sildenafil-releasing intervaginal rings were inserted into the same sheep for a period of 1 month. No sildenafil was detected in the sheep's plasma during the course of the study, however, the CVF sildenafil levels between 10³ and 10⁴ ng/gm were observed, which was higher than the median CVF sildenafil levels 1 hour after IV dosing in the same sheep and after one month of oral dosing three times a day in women.

Overall, the findings from these studies were unexpected because the CVF concentrations of the drug did not correlate with the corresponding plasma concentrations after oral dosing in humans. However, drug accumulation in the CVF secretions was apparent, which was unexpected. Typically with oral medication use, vaginal drug concentrations are somewhat dependent and much lower than plasma concentrations. After vaginal dosing in the sheep, however, no drug was detected in the plasma, but the vaginal drug concentrations were higher than those found in the above-described clinical study with symptom improvement, suggesting possible one-directional drug distribution (plasma to CVF, not CVF to plasma), which was also unexpected.

Although the preceding disclosure is focused on apparatus for treating urinary incontinence, it is noted that the disclosed intravaginal apparatuses can be used for other purposes. For example, a disclosed intravaginal apparatus can be used to treat pelvic organ prolapse or other conditions. As a further example, a disclosed intravaginal apparatus can be used as pre-surgical or post-surgical device or used before or after some form of medical treatment, such as radiation treatment. All alternative uses of the disclosed intravaginal apparatuses are deemed to fall within the scope of the present disclosure.

It is further noted that, while several of the disclosed embodiments combine a pessary with a separate drug delivery device, in some embodiments the pessary itself can be configured to release one or more drugs. In such cases, the pessaries can also be considered to comprise drug delivery devices, which can either be used alone or in combination with a further drug delivery device. When drug delivering pessaries are used, the entirety or one or more discrete portions or parts of the pessary can comprise the drug(s) to be delivered.

Claimed are:

1. An intravaginal apparatus comprising:
a drug delivery device configured for insertion into and residence within the vagina of a subject adjacent to the subject's pelvic floor, the drug delivery device further being configured to deliver one or more drugs to tissues within the vagina, the one or more drugs including a PDE5 inhibitor.

2. The apparatus of claim 1, wherein the drug delivery device is a ring.

3. The apparatus of claim 2, wherein the ring comprises a continuous toroidal body that defines an inner opening.

4. The apparatus of claim 1, further comprising a pessary configured to support the drug delivery device and to provide structural support to the pelvic organs.

5. The apparatus of claim 4, wherein the pessary comprises an inner cavity configured to receive and secure the drug delivery device.

6. The apparatus of claim 5, wherein the drug delivery device comprises a ring, the pessary comprises a continuous toroidal body that defines an inner opening, and the inner cavity is a toroidal inner cavity.

7. The apparatus of claim 6, wherein the pessary further comprises a circular channel that provides access to its toroidal inner cavity, wherein the ring can be passed through the channel to position it within the cavity.

8. The apparatus of claim 7, wherein the body of the pessary includes openings that extend to the inner cavity that enable the one or more drugs to pass from the ring to the vaginal tissues.

9. The apparatus of claim 8, wherein the circular channel is located on a first side of the pessary body and the openings are located on an opposite second side of the pessary body.

10. The apparatus of claim 9, wherein the pessary further comprises a urethral support element that extends outward from the body.

11. The apparatus of claim 10, wherein the urethral support element includes an arcuate notch.

12. The apparatus of claim 9, wherein the pessary further comprises a handle configured to be gripped during insertion and removal of the pessary.

13. The apparatus of claim 1, wherein the drug delivery device comprises a pessary configured to provide structural support to the subject's pelvic organs.

14. The apparatus of claim 13, wherein the pessary comprises a continuous body that defines an inner opening.

15. The apparatus of claim 14, wherein the pessary body comprises a continuous toroidal body that defines an inner opening.

16. The apparatus of claim 14, wherein the pessary body comprises a continuous rectangular body that defines an inner opening.

17. The apparatus of claim 16, wherein the pessary further comprises a membrane that extends across the inner opening.

18. The apparatus of claim 1, wherein the drug delivery device is configured to deliver the PDE5 inhibitor to the vaginal tissues at a rate of approximately 0.5 to 5 μg/day.

19. The apparatus of claim 1, wherein the drug delivery device is configured to deliver the PDE5 inhibitor to the vaginal tissues at a rate of approximately 0.5 to 5 mg/day.

20. A method for providing therapy to a subject, the method comprising:
inserting a drug delivery device within the vagina of the subject adjacent to the subject's pelvic floor, the drug delivery device comprising a PDE5 inhibitor;
delivering the PDE5 inhibitor to one or more of the subject's pelvic organs with the inserted drug delivery device; and
simultaneously supporting one or more of the subject's pelvic organs with the inserted drug delivery device.
